(19) United States
(12) Patent Application Publication
(10) Pub. No.: US 2020/0022645 A1
(43) Pub. Date: Jan. 23, 2020

(54) METHODS AND APPARATUS FOR SLEEP TESTING

(71) Applicant: Kyle William Miko, Coral Springs, FL (US)

(72) Inventor: Kyle William Miko, Coral Springs, FL (US)

(21) Appl. No.: 15/339,718

(22) Filed: Oct. 31, 2016

(51) Int. Cl. A61B 5/00; A61B 5/1455; A61B 90/00; G06F 19/00

(52) U.S. Cl. CPC A61B 5/4806; A61B 5/14552; A61B 5/6824; A61B 2562/226; A61B 90/361; G06F 19/3418; A61B 5/6831

(57) ABSTRACT

A user verification system verifies that a user taking a remote medical test, such as a home sleep study, is the actual user that is supposed to be taking the test. One issue in home sleep studies is that it can be difficult to confirm that the user that wore the home sleep monitor is actually the user that was supposed to take the test. The user verification system includes a wrist strap that, once attached to the user, cannot be removed without being noted in the home sleep study results. A probe connector can connect to the wrist strap in such a manner that removal of the probe connector from the wrist strap is noted in the home sleep study results. The probe connector can connect to a probe, such as a pulse oximeter, and can connect to a recording device that logs the data received from the probe.

### BACKGROUND OF THE INVENTION

### 1. Field of the Invention

One or more embodiments of the invention relates generally to medical devices. More particularly, the invention relates to methods and apparatus to ensure chain of custody for home sleep testing, occupational/Department of Transportation health testing and the like.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

A study conducted by the University of Pennsylvania and sponsored by the Federal Motor Carrier Safety Administration (FMCSA) and the American Transportation Research Institute of the American Trucking Associations found that almost one-third (28 percent) of commercial truck drivers have mild to severe sleep apnea.

Sleep apnea is a breathing-related sleep disorder that causes brief interruptions of breathing during sleep. These pauses in breathing can last at least 10 seconds or more and can occur up to 400 times a night. Sleep apnea is a serious, potentially life-threatening condition that often goes unrecognized and undiagnosed. Because sleep apnea affects one's sleep, it also affects one's daytime alertness and performance. Untreated sleep apnea can make it difficult for a person to stay awake, focus their eyes, and react quickly while driving. In general, studies show that people with untreated sleep apnea have an increased risk of being involved in a fatigue-related motor vehicle crash.

While FMCSA regulations do not specifically address sleep apnea, they do prescribe that a person with a medical history or clinical diagnosis of any condition likely to interfere with their ability to drive safely cannot be medically qualified to operate a commercial motor vehicle (CMV) in interstate commerce. However, once successfully treated, a driver may regain their “medically-qualified-to-drive” status. It is important to note that most cases of sleep apnea can be treated successfully.

To show successful treatment, and thus to be medically qualified to drive, a person must prove their successful treatment, often requiring one or more stays at a sleep study center. This can be costly and inconvenient to the user, however. Therefore, home testing is often preferred. However, establishing chain of custody, that is, proof that the test data obtained in home testing belongs to the test subject, can be difficult.

In view of the foregoing, there is a need for methods and systems that can provide home sleep testing to individuals while ensuring proper chain of custody for the medical examiner reviewing the test results and, for example, issuing a medically qualified to drive status to a person.

### SUMMARY OF THE INVENTION

Embodiments of the present invention provide a user verification system for a health study on a patient comprising a wrist strap fitting onto the patient; at least two electrical connectors disposed on a top side of the wrist strap; a conductive member disposed along a length of the wrist strap, the conductive members electrically connecting the at least two electrical connectors; a verification device having device electrical connectors mating with the at least two electrical connectors of the wrist strap; and a lead connected to the verification device and extendable to connect to a study device, wherein the study device is adapted to detect when the electrical connectors of the verification device are mated with the at least two electrical connectors of the wrist strap.

Embodiments of the present invention further provide a user verification system for a health study on a patient comprising a wrist strap fitting onto the patient; at least two electrical connectors disposed on a top side of the wrist strap; a conductive member disposed along a length of the wrist strap, the conductive members electrically connecting the at least two electrical connectors, wherein the conductive member extends at least once to each longitudinal end of the wrist strap; a verification device having device electrical connectors mating with the at least two electrical connectors of the wrist strap; a lead connected to the verification device and extendable to connect to a study device; a lead extending from the verification device to a study probe; and a connector operable to non-removably connect the wrist strap to the patient, wherein the study device is adapted to detect when the electrical connectors of the verification device are mated with the at least two electrical connectors of the wrist strap.

Embodiments of the present invention also provide a method for performing a health study on a patient comprising delivering, to the patient, a user verification system comprising a wrist strap fitting onto the patient; at least two electrical connectors disposed on a top side of the wrist strap; a conductive member disposed along a length of the wrist strap, the conductive members electrically connecting the at least two electrical connectors; and a verification device having device electrical connectors mating with the at least two electrical connectors of the wrist strap; verifying the wrist strap is attached to the patient; connecting a study probe to the verification device; and connecting a study device to the verification device, wherein the study device is adapted to detect when the electrical connectors of the verification device are mated with the at least two electrical connectors of the wrist strap.

In some embodiments, the study probe is a pulse oximeter probe. In some embodiments, an indication is provided to the patient when the verification device is properly connected to the wrist strap.

In some embodiments, a video signal is sent to a server, the video signal including video showing the patient applying the wrist strap to themselves, thereby confirming the proper patient is wearing the wrist strap.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 is a perspective view of a user wearing a sleep testing device secured to the user via a wrist strap with a user verification device according to an exemplary embodiment of the present invention;

FIG. 2 is a perspective view of the user of FIG. 1, illustrating detachment of the user verification device from the wrist strap;

FIG. 3 is a back view of the wrist strap of FIG. 1;

FIG. 4 is a perspective view of the user verification device of FIG. 1, disconnected from the wrist strap;

FIG. 5 is a perspective view of the user verification device of FIG. 1, connected to the wrist strap;

FIG. 6 is a perspective view of a user verification device according to another exemplary embodiment of the present invention; and

FIG. 7 is a schematic representation of a confirmation system to ensure the appropriate user attaches the user verification device, according to an exemplary embodiment of the present invention.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary, a variety of optional components are described to illustrate the wide variety of possible embodiments of the present invention.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention relate to a user verification system for verifying a user taking a remote medical test, such as a home sleep study, is the actual user that is supposed to be taking the test. One issue in home sleep studies is that it can be difficult to confirm that the user that wore the home sleep monitor is actually the user that was supposed to take the test. The user verification system includes a wrist strap that, once attached to the user, cannot be removed without being noted in the home sleep study results. A probe connector can connect to the wrist strap in such a manner that removal of the probe connector from the wrist strap is noted in the home sleep study results. The probe connector can connect to a probe, such as a pulse oximeter, and can connect to a recording device that logs the data received from the probe, via the probe connector attached to the wrist strap.

Thus, once it is confirmed that the user is wearing the wrist strap, one can confirm, with relatively certainty, that the results obtained from a sleep study are the results from the person wearing the wrist strap. In some embodiments, the user may apply the wrist strap themselves, where a video telemedicine system can be employed so that a medical professional can monitor the proper application of the wrist strap to the proper person.

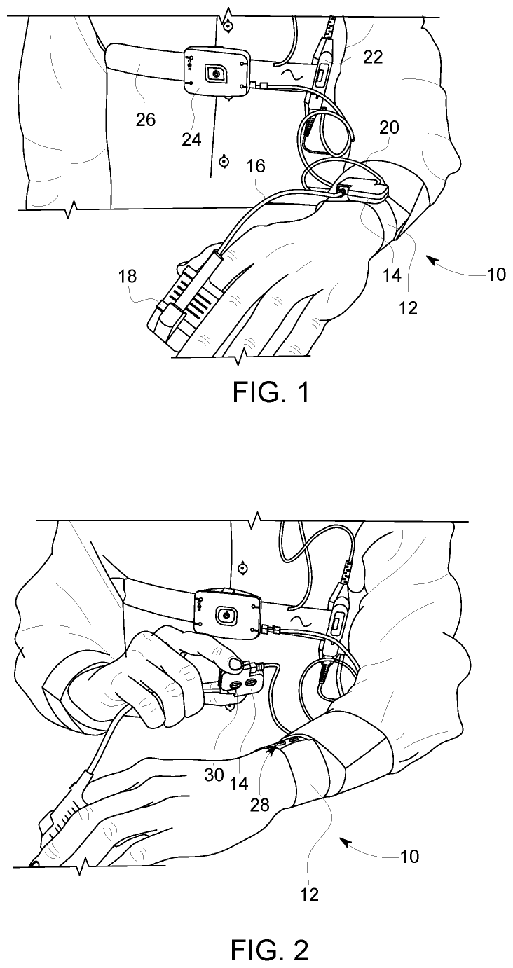

Referring to FIGS. 1 through 5, a user verification device 10 includes a wrist strap 14 that may be applied to a patient's wrist, as shown. A verification connector 14 can include metallic connectors 30 that connect with wrist strap connectors 28 disposed on the wrist strap 14.

The verification connector 14 can include an analytical tool lead 16 that terminates in an analytical tool, such as a pulse oximeter finger probe 18. The pulse oximeter finger probe 18 can be, for example, a disposable probe that plugs into the verification connector 14. A study device 24 may connect to the verification connector 14 via a study device lead 20. In some embodiments, an intermediate connector 22 may be disposed between the verification connector 14 and the study device 24. The study device 24 may, for example, strap about the patient's torso with a strap 26. Of course, other connections between the verification connector 14 and the study device 24, as well as other attachments to the patient are contemplated within the scope of the present invention.

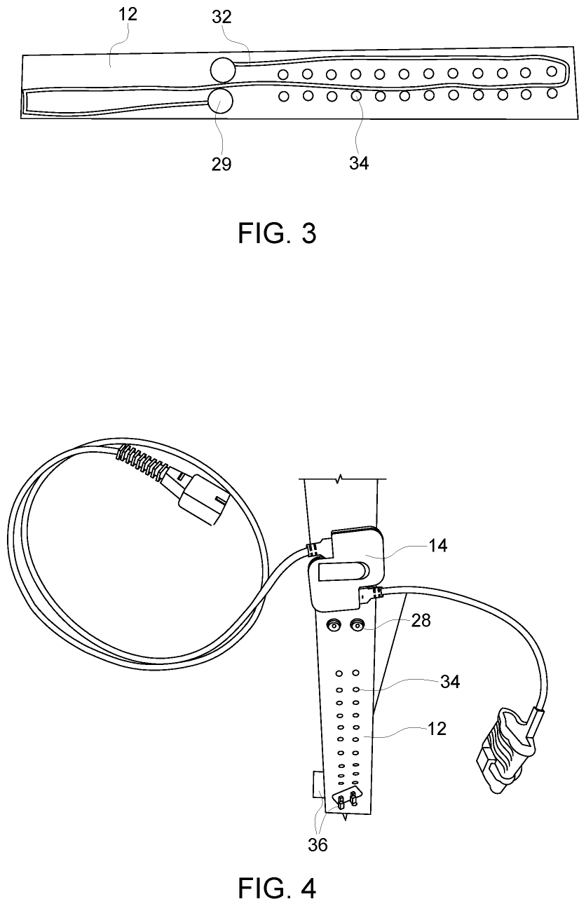

Referring specifically to FIGS. 2 and 3, the wrist strap 14 can include an electrically conductive portion 32 that runs along the length of the wrist strap 12 and may connect to terminals 29 of the wrist strap connectors 28. Thus, there is electrically continuity between the two wrist strap connectors 28. Once the wrist strap 14 is applied to the patient, the conductive portion 32 prohibits removal of the wrist strap 12 without discontinuing the electrical continuity between the wrist strap connectors 28. Accordingly, the study device 24 may record when the metallic connectors 30 of the verification connector 14 are conductive therebetween and may record if the verification connector 14 is removed from the wrist strap 12 or if the wrist strap 12 is cut or otherwise removed from the patient.

In some embodiments, the conductive portion 32 may have a specific, predefined resistance that is sensed by the study device 24 by measuring the resistance between the metallic connectors 30 of the verification connector 14. In this embodiment, the patient may not simply remove the verification connector 14 and jump the metallic connectors together to permit another user to complete the study, as the study device 24 would have the ability to detect whether the predetermined resistance is present in the conductive portion 32. Moreover, should the user attempt to stretch the wrist band 12 in efforts to remove it, this may lower the resistance of the conducive portion 32, which would be detected by the study device 24.

The study device 24 may include one or more indicator, such as an indicator light, that can visually show the patient when the verification connector 14 is attached to the wrist strap 12. Such indicator may, for example, illuminate in different colors to show proper connections of the study device 24 to the study probe 18, via the verification connector 14. The data recorded on the study device 24 may show any changes in connection status.

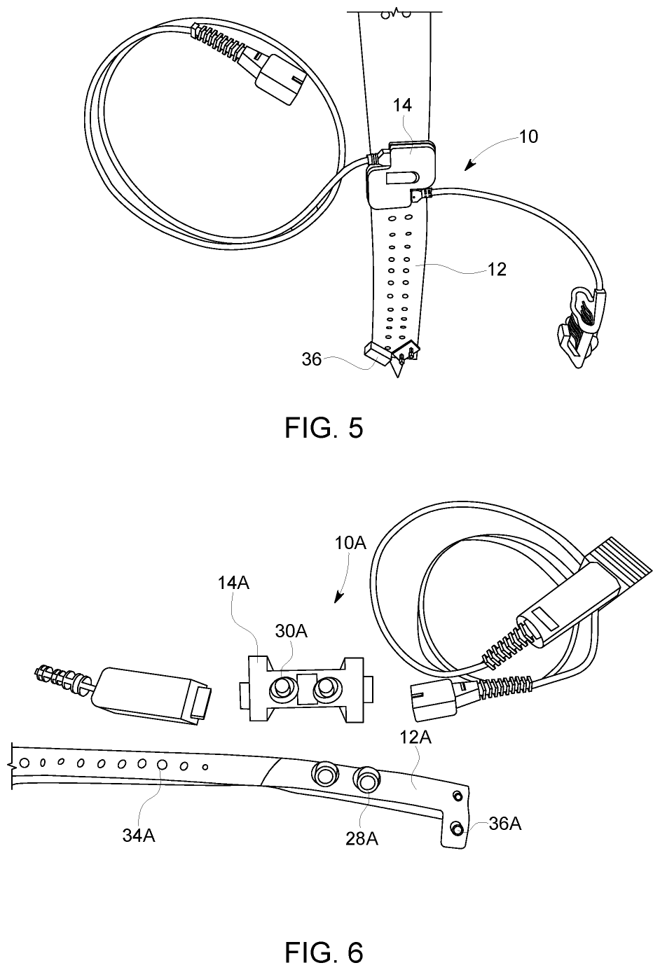

Referring now specifically to FIGS. 4 and 5, the wrist band 12 may include a plurality of holes 34 along its length. A closure 36 may fit through the holes to fix the wrist band onto the patient's wrist. The closure 36 may be non-removable once connected and may securely apply the wrist band 12 to the patient. While one type of closure 36 is shown in FIGS. 5 and 6, various types of connectors, closures and joining elements may be used within the scope of the present invention.

Referring to FIG. 6, in an alternate embodiment of the present invention, a verification device 10A can include a wrist band 12A which may include wrist band connectors 28A that mate with metallic connectors 30A on an underside of a verification connector 14A. In this embodiments, the wrist band 12A may include a single row of holes 34A that are connected with a closure 36A.

The features discussed above help ensure chain of custody for studies, such as sleep studies. The above features address the issue of assuring the study is performed by the person wearing the wrist band. However, one must also be assured that the person wearing the band, when applied, is the proper person for the study.

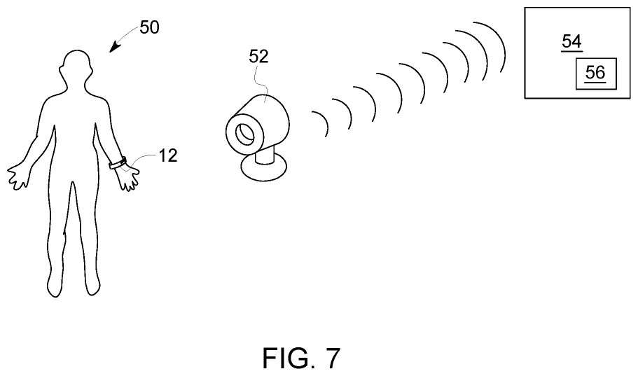

To address this issue, referring now to FIG. 7, a user 50 may receive a study kit from a physician, in the mail, or the like. The user 50 may use a camera, such as a web cam 52 to send a signal to a monitoring station 54 which may include memory 56 for storing video data. The user 50 may apply the wrist strap 12 while video is being recorded and saved to verify that the user applying the band is the desired person to be studied. Of course, in some embodiments, the wrist band 12 may simply be applied by a physician, a study coordinator, or the like, who may attest that the proper person was wearing the band. Because the wrist band 12 contains no wires or cords extending therefrom, the wrist band may be applied well prior to the desired study time without interfering with the user's normal activities.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims. Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

1. A user verification system for a health study on a patient, comprising:
a wrist strap fitting onto the patient;
at least two electrical connectors disposed on a top side of the wrist strap;
a conductive member disposed along a length of the wrist strap, the conductive members electrically connecting the at least two electrical connectors;
a verification device having a housing with integrated device electrical connectors mating with the at least two electrical connectors of the wrist strap;
a lead connected to the verification device and extendable to connect to a study device; and
an analytical tool lead connected to the verification and extendable to an analytical tool, wherein
the study device is adapted to detect when the electrical connectors of the verification device are mated with the at least two electrical connectors of the wrist strap.

2. (canceled)

3. The user verification system of claim 1, wherein the analytical tool is a pulse oximeter probe.

4. The user verification system of claim 1, further comprising a connector operable to non-removably connect the wrist strap to the patient.

5. The user verification system of claim 1, wherein the conductive member extends at least once to each longitudinal end of the wrist strap.

6. The user verification system of claim 1, wherein the study device provides an indication to the patient when the verification device is properly connected to the wrist strap.

7. The user verification system of claim 1, further comprising a web cam for sending a video signal to a server, the video signal including video showing the patient applying the wrist strap to themselves.

8. A user verification system for a health study on a patient, comprising:
a wrist strap fitting onto the patient;
at least two electrical connectors disposed on a top side of the wrist strap;
a conductive member disposed along a length of the wrist strap, the conductive members electrically connecting the at least two electrical connectors, wherein the conductive member extends at least once to each longitudinal end of the wrist strap;
a verification device having a housing with integrated device electrical connectors mating with the at least two electrical connectors of the wrist strap;
a lead connected to the verification device and extendable to connect to a study device;
a lead extending from the verification device to a study probe; and
a connector operable to non-removably connect the wrist strap to the patient, wherein
the study device is adapted to detect when the electrical connectors of the verification device are mated with the at least two electrical connectors of the wrist strap.

9. The user verification system of claim 8, wherein the study probe is a pulse oximeter probe.

10. The user verification system of claim 8, wherein the study device provides an indication to the patient when the verification device is properly connected to the wrist strap.

11. The user verification system of claim 8, further comprising a web cam for sending a video signal to a server, the video signal including video showing the patient applying the wrist strap to themselves.

12. A method for performing a health study on a patient, comprising:
delivering, to the patient, a user verification system comprising:
a wrist strap fitting onto the patient;
at least two electrical connectors disposed on a top side of the wrist strap;
a conductive member disposed along a length of the wrist strap, the conductive members electrically connecting the at least two electrical connectors;
a verification device having a housing with integrated device electrical connectors mating with the at least two electrical connectors of the wrist strap;
a lead connected to the verification device and extendable to connect to a study device; and
an analytical tool lead connected to the verification and extendable to an analytical tool;
verifying the wrist strap is attached to the patient;
connecting a study probe to the verification device; and
connecting a study device to the verification device, wherein
the study device is adapted to detect when the electrical connectors of the verification device are mated with the at least two electrical connectors of the wrist strap.

13. The method of claim 12, wherein the study probe is a pulse oximeter probe.

14. The method of claim 12, wherein the conductive member extends at least once to each longitudinal end of the wrist strap.

15. The method of claim 12, further comprising providing an indication to the patient when the verification device is properly connected to the wrist strap.

16. The method of claim 12, the step of verifying the wrist strap is attached to the patient includes sending a video signal to a server, the video signal including video showing the patient applying the wrist strap to themselves.
